(19) United States
(12) Patent Application Publication
(10) Pub. No.: US 2023/0038188 A1
(43) Pub. Date: Feb. 9, 2023

(54) PHARMACEUTICAL COMPOSITION AND HEALTH FUNCTIONAL FOOD FOR PREVENTING, AMELIORATING OR TREATING THROMBOSIS CONTAINING EXTRACT OF VESPA VELUTINA NIGRITHORAX AS ACTIVE INGREDIENT

(71) Applicant: Andong National University Industry-Academic coorperation Foundation, Gyeongsangbuk-do (KR)

(72) Inventor: Ho Yong SOHN, Daegu (KR)

(21) Appl. No.: 17/553,491

(22) Filed: Dec. 16, 2021

(30) Foreign Application Priority Data
Jul. 27, 2021 (KR) 10-2021-0098673

(51) Int. Cl. A61K 35/64; A61P 7/02; A23L 33/10; A23L 33/00

(52) U.S. Cl. CPC A61K 35/64; A61P 7/02; A23L 33/10; A23L 33/40; A23V 2002/00

(57) ABSTRACT

An antithrombotic composition containing an extract of Vespa velutina nigrithorax as an active ingredient, and more particularly, a pharmaceutical composition and a health functional food for preventing, ameliorating or treating thrombosis by inhibition of blood coagulation and inhibition of platelet aggregation, which contain an ethyl acetate fraction of an extract of Vespa velutina nigrithorax as an active ingredient. The extract exhibits antithrombotic activity by inhibiting platelet aggregation while exhibiting strong anticoagulant activity by inhibiting thrombosis-related enzymes and blood clotting factors. Also, the extract has no human erythrocyte hemolytic activity, has excellent heat stability, and the thrombosis-related enzyme and blood clotting factor inhibitory effects thereof are not lost even under an acidic condition of pH 2 and in plasma.

### BACKGROUND

The present disclosure relates to an antithrombotic composition containing an extract of Vespa velutina nigrithorax (wasp) as an active ingredient, and more particularly, to a pharmaceutical composition and a health functional food for preventing, ameliorating or treating thrombosis by inhibition of blood coagulation and inhibition of platelet aggregation, which contain an ethyl acetate fraction of an extract of Vespa velutina nigrithorax as an active ingredient.

Blood as a component of the human body has various important functions such as transport of oxygen, nutrients and waste products, buffering action, body temperature maintenance, osmotic pressure regulation, ion balance maintenance, constant water content maintenance, humoral regulation, maintenance and regulation of blood pressure, and host defense. It has been reported that normal blood circulation is facilitated as the blood coagulation system and the thrombolytic system in vivo are controlled complementarily to each other to facilitate blood circulation, and in the mechanism of the blood coagulation system among these systems, platelets adhere to blood vessel walls and aggregate to form platelet thrombi, and then the blood coagulation system is activated and fibrin thrombi are formed with respect to platelet aggregates.

In production of fibrin thrombi, thrombin that is involved in fibrin coagulation are activated by multi-step reactions of many blood coagulation factors to produce fibrin monomers from fibrinogen, and the fibrin monomers are polymerized by calcium to form fibrin polymers that bind to platelets and endothelial cells and that are crosslinked by XIII factor, thereby producing permanent thrombi. In addition, thrombin plays a pivotal role in thrombus formation, such as promoting blood clotting by activating platelets, factor V and factor VII. Thus, a substance that inhibits thrombin activity may be used as a very useful preventive and therapeutic agent against various thrombotic diseases that are caused by excessive blood clotting abnormalities. It is known that prothrombin activation following the sequential activation of factor XII, factor XI, factor IX and factor X in the extrinsic pathway of thrombosis finally activates thrombin. Thus, specific inhibition of blood coagulation factors also becomes an important target for the development of agents for treating thrombotic diseases. For the intrinsic pathway of thrombosis, thrombosis following the activation of factor II (prothrombin), factor V, factor VII and factor X is known. Until now, various anticoagulants, anti-platelet agents or thrombolytic agents, including heparin, coumarin, aspirin, urokinase and the like, have been used for the prevention and treatment of thrombotic diseases. However, these agents are very expensive, and the use thereof is limited due to hemorrhagic side effects, gastrointestinal disorder and hypersensitivity.

Meanwhile, wasps are insects belonging to the family Vespidae of the order Hymenopterathe, and are predatory insects that eat bees and other small insects. There are about 30 wasp species of 5 genera (Vespa, Vespula, Dolichovespula, Parapolybia, and Polistes) worldwide. In Korea, the following 10 wasp species are known: Vespa analis parallela Andre, V. baslis Smith, V. ducalis Smith, V. dybowskii Andre, Vespa crabro crabroniformis Smith, V. crabro flavofasciata Cameron, V. mandarinia Smith, V. simillima simillima Smith, V. smillima xanthoptera Cameron, and V. velutina nigrithorax. Wasps have different habitats and distributions depending on the species, build wasp nests and inhabit in groups mainly in low mountains, treetops or in the ground.

Wasps are also responsible for pollination and perform the roles of parasites and predators. In particular, some wasps are recognized as pests in the beekeeping industry because they prey on adult honeybees, and damage by them is gradually increasing. In recent years, wasps have been a threat to humans and animals due to wasp stings, and multiple stings with the powerful wasp venom can sometimes cause death. Thus, in order to prevent damage caused by wasps, studies on wasp attraction and capture (Jung Jun-Seong et al., 2018. Trends in Agriculture & Life Sciences 56: 35-45) and the control of wasps using a specific frequency (Kim et al., 2019. J. Apiculture 34: 7-13) have been conducted.

In Korea, wasps have been recognized as a nourishing tonic food since ancient times, and as a representative example, wasp wine made by soaking wasps in alcohol has been used for the treatment of inflammation, epilepsy, convulsions and dental diseases (Heo Jun's Donguibogam, 1615). However, studies on wasps are very limited, and most of the existing studies are studies on ecology and classification of wasps (Murat et al., 2016, Carbohydrate Polymers 145: 64-70) and wasp venom (Xinwang et al., 2013, Toxicon 74: 151-157: Yoon et al., 2015, Journal of Asia-Pacific Entomology 18: 815-823), and there are no studies on the useful physiological activity of wasps themselves.

A study on wasp venom has reported the strong hemolytic activity and cytotoxicity of the wasp venom, and recently, anti-inflammatory activity, allergy induction and neurotoxicity by histamine, serotonin, phospholipase A2, hyaluronidase, etc. have been found (Lee, B. H., Park, H. J. 1998. J. Korean Environ. Sci. Sco. 62-66; Sabe et al., 2017. Med. Inflamm. 6978194. doi: 10.1155/2017/6978194). In addition, damage caused by acute thrombosis after a wasp sting has been continuously reported (Chen, D M. et al., 2004. The American journal of medicine 116: 567-569; Min J H et al., 2013. Korean circulation journal 43: 561-564). However, until now, there has been no report on the antithrombotic activity of a wasp extract.

Patents related to wasps include Korean Patent No. 10-2028363 [entitled “System for combating harmful wasps”] and Korean Patent No. 10-2075057 [entitled “Wasp trapping device capable of collecting wasps alive”], Korean Patent No. 10-1972070 [entitled “Composition for preventing or treating gout containing bee venom isolated from worker bees of V. mandarinia Smith”], Korean Patent No. 10-1972074 [entitled “Composition for preventing or treating Alzheimer's disease containing bee venom isolated from worker bees of V. mandarinia Smith”], Korean Patent No. 10-1374327 [entitled “Functional cosmetic composition containing V. mandarinia Smith venom extract”], Korean Patent Application Publication No. 10-1999-0039050 [entitled “Agent for preventing and treating acne containing wasp venom extract”], Korean Patent Application Publication No. 10-2012-0111206 [entitled “Method of preparing functional cosmetic composition using wasps”], Korean Patent Application Publication No. 10-2012-0100450 [entitled “Method of preparing functional cosmetic composition using V. mandarinia Smith], and Korean Patent Application Publication No. 10-2005-0028992 [“Method of producing traditional wine using wasps and traditional wine liquor using wasps produced thereby”]. However, until now, there has been no known patent related to the antithrombotic activity of a wasp extract.

Prior Art Documents: 1) Chen, D M. et al., 2004. The American journal of medicine 116: 567-569; and 2) Min J H et al., 2013. Korean circulation journal 43: 561-564

### SUMMARY OF THE INVENTION

The present disclosure has been made in order to solve the above-described problems occurring in the prior art, and an object of the present disclosure is to provide an antithrombotic composition containing an extract of Vespa velutina nigrithorax as an active ingredient.

To achieve the above object, the present disclosure provides a pharmaceutical composition for preventing or treating thrombosis containing an extract of Vespa velutina nigrithorax as an active ingredient.

The extract is preferably an ethanol extract.

The extract is preferably an ethyl acetate fraction obtained after ethanol extraction.

The present disclosure also provides a health functional food for preventing or ameliorating thrombosis containing an extract of Vespa velutina nigrithorax as an active ingredient.

The extract is preferably an ethanol extract.

The extract is preferably an ethyl acetate fraction obtained after ethanol extraction.

### BRIEF DESCRIPTION OF THE DRAWINGS

FIG. 1 shows photographs of V. mandarinia Smith, V. simillima simillima Smith and Vespa velutina nigrithorax used in the Examples of the present disclosure.

FIG. 2 shows the human platelet aggregation inhibitory activities of a Vespa velutina nigrithorax extract and sequential organic solvent fractions thereof. 1: a solvent control (DMSO), 2: aspirin (0.25 mg/ml), 3: an ethanol extract (0.25 mg/ml) of Vespa velutina nigrithorax, 4: a hexene fraction (0.25 mg/ml) of the Vespa velutina nigrithorax extract, 5: an ethyl acetate fraction (0.25 mg/ml) of the Vespa velutina nigrithorax extract, 6: a butanol fraction (0.25 mg/ml) of the Vespa velutina nigrithorax extract, and 7: water residue (0.25 mg/ml) after organic solvent fractionation of the Vespa velutina nigrithorax extract.

### DETAILED DESCRIPTION OF THE INVENTION

Hereinafter, the present disclosure will be described in detail.

In order to test the antithrombotic efficacy of wasps, the present inventors first collected V. mandarinia Smith, V. simillima simillima Smith and Vespa velutina nigrithorax in Korea, and then washed the collected wasps with water to remove foreign substances, followed by soaking in 30% ethanol and 1 year of leaching. Thereafter, the extracts were filtered and concentrated under reduced pressure, and the Vespa velutina nigrithorax extract was recovered as an antithrombotic active ingredient. It was confirmed that the active ingredient is characterized by having excellent heat stability and acid stability. Thus, the active ingredient was intended to be used as an active ingredient for a pharmaceutical composition and a health functional food for preventing, ameliorating or treating thrombosis.

Specifically, in order to develop a pharmaceutical composition and a health functional food for preventing, ameliorating or treating thrombosis by using wasps known to have anti-inflammatory and antioxidant effects, the present inventors prepared ethanol extracts of three wasp species and evaluated the antithrombotic activities of these extracts by direct inhibitory activity against human thrombin (thrombin time), inhibitory activity against prothrombin (prothrombin time), and activated partial thromboplastin time (aPTT), thereby confirming that the Vespa velutina nigrithorax extract has excellent antithrombotic activity.

Accordingly, sequential organic solvent fractions of the Vespa velutina nigrithorax extract were prepared, and then the antithrombotic activity of each of the organic solvent fractions was evaluated. As a result, it was confirmed that an ethyl acetate fraction of the extract has strong anticoagulant activity together with excellent platelet aggregation inhibitory activity. In particular, the ethyl acetate fraction of the Vespa velutina nigrithorax extract exhibited anticoagulant activity in a concentration-dependent manner, and extended the thrombin time, the prothrombin time and the aPTT time by 15 times or more, 4.1 times and 3.29 times, respectively, at a concentration of 5 mg/ml, and extended all the thrombin time, the prothrombin time and the aPTT time by 15 times or more at a concentration of 6 mg/ml. In addition, it was confirmed that the ethyl acetate fraction showed little or no hemolytic activity for human erythrocytes, suggesting that it causes no acute toxicity.

Therefore, the present disclosure provides a pharmaceutical composition for preventing or treating thrombosis containing an extract of Vespa velutina nigrithorax as an active ingredient.

The extract is preferably an ethanol extract.

The extract is preferably an ethyl acetate fraction obtained after ethanol extraction.

The present disclosure also provides a health functional food for preventing or ameliorating thrombosis containing an extract of Vespa velutina nigrithorax as an active ingredient.

The extract is preferably an ethanol extract.

The extract is preferably an ethyl acetate fraction obtained after ethanol extraction.

Hereinafter, a method for preparing the Vespa velutina nigrithorax extract of the present disclosure and a test for the efficacy of the Vespa velutina nigrithorax extract will be described in more detail.

In order to achieve the object of the present disclosure, the present inventors performed an experimental method including steps of: collecting three wasp species; preparing wasp extracts; preparing sequential organic solvent fractions (hexene, ethyl acetate and butanol fractions) from each of the wasp extracts and then preparing water residue; evaluating the antithrombotic activities of the extracts and the fractions; and examining the stability of each ethyl acetate fraction as an active ingredient.

The Vespa velutina nigrithorax extract contained in the composition of the present disclosure may be obtained by a method including steps of: extracting Vespa velutina nigrithorax with 30% ethanol for about 1 year; and filtering the extract through a filter having a mesh size of 0.06 mm or less and concentrating the filtrate under reduced pressure.

A solvent that is used in the present disclosure may be water (cold or hot water), spirit, an anhydrous or hydrous lower alcohol having 1 to 4 carbon atoms (e.g., methanol, ethanol, spirit, propanol, butanol, etc.), or a mixed solvent of the lower alcohol and water, and hot-water extraction or ethanol extraction is most preferred.

In a preferred embodiment of the present disclosure, Vespa velutina nigrithorax may be extracted with ethanol. In addition, the ethanol extract may be fractionated sequentially with organic solvents (hexene, ethyl acetate and butanol) or fractioned with each of the organic solvents to additionally obtain a hexene fraction, an ethyl acetate fraction, a butanol fraction and water residue.

The Vespa velutina nigrithorax extract of the present disclosure may be prepared into powder through a conventional powdering process such as vacuum drying, freeze-drying, or spray-drying. The extract is not degraded by various degrading enzymes in plasma, and remains active even upon heat treatment at 100° C. and in the human stomach at pH 2.

The active ingredient of the present disclosure may be used for the prevention or treatment of various diseases associated with thrombosis. The diseases include, for example, arterial thromboses, such as acute myocardial infarction, chest pain, difficulty in breathing, loss of consciousness, ischemic stroke, hemorrhagic stroke, headache, dyskinesia, paresthesia, personality change, blurred vision, epileptic seizures, pulmonary thrombosis, deep vein thrombosis, lower extremity edema, pain, and acute peripheral arterial occlusion, as well as venous thromboses, such as deep vein thrombosis, portal vein thrombosis, acute renal vein occlusion, cerebral vein sinus thrombosis, and central retinal vein occlusion.

In a preferred embodiment, the pharmaceutical composition of the present disclosure may be used as an anticoagulant (blood clotting inhibitor) or an antiplatelet agent (platelet aggregation inhibitor).

For use, the pharmaceutical composition containing the active ingredient of the present disclosure may be formulated in various forms, including oral dosage forms such as powders, granules, tablets, capsules, suspensions, emulsions, syrups, and aerosols, or injection dosage forms sterile injections, according to conventional methods depending on the intended use thereof, and may be administered through various routes including oral, intravenous, intraperitoneal, subcutaneous, rectal and topical routes.

This pharmaceutical composition may further contain a carrier, excipient or diluent. Examples of a carrier, excipient or diluent that may be contained in the pharmaceutical composition include lactose, dextrose, sucrose, sorbitol, mannitol, xylitol, erythritol, maltitol, starch, gum acacia, alginate, gelatin, calcium phosphate, calcium silicate, cellulose, methyl cellulose, amorphous cellulose, polyvinyl pyrrolidone, water, methylhydroxybenzoate, propylhydroxybenzoate, talc, magnesium stearate, and mineral oil. In addition, the pharmaceutical composition of the present disclosure may further contain a filler, an anti-aggregating agent, a lubricant, a wetting agent, a flavoring agent, an emulsifying agent, a preservative, and the like.

In a preferred embodiment, solid formulations for oral administration include tablets, pills, powders, granules, capsules, and the like, and such solid formulations are prepared by mixing the pharmaceutical composition with at least one excipient, for example, starch, calcium carbonate, sucrose, lactose, gelatin, and the like. In addition to simple excipients, lubricants such as magnesium stearate, talc and the like may also be used.

In a preferred embodiment, liquid formulations for oral administration include, for example, a suspension, an internal solution, an emulsion, a syrup, and the like. In addition to water and liquid paraffin, which are commonly used simple diluents, various excipients, for example, a wetting agent, a sweetening agent, a flavoring agent, a preservative, and the like may be contained.

In a preferred embodiment, formulations for parenteral administration include, for example, sterile aqueous solutions, non-aqueous solutions, suspensions, emulsions, lyophilized formulations, suppositories, and the like. As non-aqueous solvents or suspending agents, there may be used propylene glycol, polyethylene glycol, vegetable oil such as olive oil, injectable ester such as ethyl oleate, and the like. Injections may contain conventional additives such as a solubilizing agent, an isotonic agent, a suspending agent, an emulsifying agent, a stabilizing agent, a preservative, and the like.

The active ingredient of the present disclosure is administered in a pharmaceutically effective amount. As used herein, the term “pharmaceutically effective amount” refers to an amount sufficient to treat a disease at a reasonable benefit/risk ratio applicable to any medical treatment. The effective dose level of the active ingredient may be determined depending on factors, including the kind and severity of the patient's disease, the activity of the drug, sensitivity to the drug, the time of administration, the route of administration, excretion rate, the duration of treatment, and drugs used in combination with the composition, as well as other factors well known in the medical field. The pharmaceutical composition of the present disclosure may be administered individually or in combination with other therapeutic agents, and may be administered sequentially or simultaneously with conventional therapeutic agents. The pharmaceutical composition may be administered in a single or multiple dosage form. It is important to administer the pharmaceutical composition in the minimum amount that can exhibit the maximum effect without causing side effects, in view of all the above-described factors, and this amount can be easily determined by a person skilled in the art.

In a preferred embodiment, the effective amount of the active ingredient in the pharmaceutical composition of the present disclosure may vary depending on the patient's age, sex and body weight. Generally, the active ingredient may be administered daily or every other day at a dose of 1 to 5,000 mg/kg body weight, preferably 100 to 3,000 mg/kg body weight, or may be administered 1 to 3 times a day at this dose. However, since the dose may increase or decrease depending on the route of administration, the severity of the disease, the patient's sex, weight and age, etc., the dose is not intended to limit the scope of the present disclosure in any way.

The pharmaceutical composition of the present disclosure may be administered to a subject through various routes. All modes of administration can be contemplated, and for example, the composition may be administered orally, intrarectally, or by intrarectal, intravenous, intramuscular, subcutaneous, intrauterine, intrathecal or intracerebroventricular injection.

In the present disclosure, the term “administration” means providing a given substance to a patient by any suitable method. The pharmaceutical composition of the present disclosure may be administered orally or parenterally through all general routes as long as it can reach the target tissue. In addition, the composition of the present disclosure may also be administered using any device capable of delivering the active ingredient to target cells.

In the present disclosure, the term “subject” is not particularly limited, but includes, for example, humans, monkeys, cattle, horses, sheep, pigs, chicken, turkeys, quails, cats, dogs, mice, rats, rabbits or guinea pigs, and preferably refers to mammals, more preferably humans.

In addition, the health functional food of the present disclosure may be variously used in foods and beverages that are effective in preventing and ameliorating thrombosis. Foods containing the active ingredient of the present disclosure include various foods, for example, beverages, gums, teas, vitamin complexes, health supplement foods and the like, and may be used in the form of powders, granules, tablets, capsules or beverages.

The active ingredient of the present disclosure may generally be added in an amount of 0.01 to 15 wt % based on the total food weight. For a health beverage composition, the active ingredient may be added in an amount of 0.02 to 10 g, preferably 0.3 to 1 g, based on 100 ml of the health beverage composition.

The health functional food of the present disclosure may additionally contain food-acceptable additives, for example, natural carbohydrates and various flavoring agents, in addition to containing the Vespa velutina nigrithorax extract as an essential component at the indicated percentage.

Examples of the natural carbohydrates include conventional sugars, such as monosaccharides (e.g., glucose, fructose, etc.), disaccharides (e.g., maltose, sucrose, etc.), polysaccharides (e.g., dextrin, cyclodextrin, etc.), and sugar alcohols such as xylitol, sorbitol, erythritol or the like.

Examples of the flavoring agents that may be used in the present disclosure include thaumatin, rebaudioside A, glycyrrhizin, saccharin, aspartame, etc. The flavoring agent is used in an amount of about 1 to 20 g, preferably about 5 to 12 g, based on 100 mL of the health functional food of the present disclosure. In addition, the health functional food of the present disclosure may contain various nutrients, vitamins, minerals, flavoring agents such as synthetic flavoring agents and natural flavoring agents, colorants, extenders, pectic acid and its salt, alginic acid and its salt, organic acids, protective colloidal thickeners, pH adjusting agents, stabilizers, preservatives, glycerin, alcohol, carbonizing agents that are used in carbonated beverages, etc. Additionally, the health functional food of the present disclosure may contain fruit flesh that is used for the preparation of natural fruit juice, fruit juice beverages or vegetable beverages. These components may be used individually or in combination. The content of these additives is generally selected in the range of 0.01 to about 20 parts by weight based on 100 parts by weight of the health functional food.

Hereinafter, the present disclosure will be described in more detail with reference to specific examples. The following examples merely describe one preferred embodiment of the present disclosure, and the scope of the present disclosure is not limited by the contents described in the following examples.

### Example 1: Wasp Collection and Wasp Extract Preparation

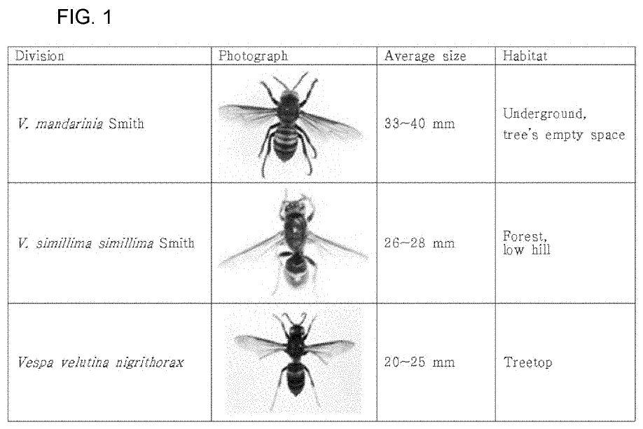

In August 2019, V. mandarinia Smith, V. simillima simillima Smith and Vespa velutina nigrithorax were collected in Gyeongsangbuk-do, Korea, and each wasp species was identified by an insect expert, Andong University professor Jeong Cheol-Hee (FIG. 1). The collected wasps were soaked in 30% ethanol without separate pretreatment and extracted therein for about 1 year. In this case, about 5 liters of 30% ethanol was added to 100 wasps. Thereafter, the wasps were removed, and the wasp extracts were filtered, and dried under reduced pressure and recovered.

### Example 2: Analysis of Components of Wasp Extracts

The total polyphenol, total flavonoid, total sugar and reducing sugar contents in each of the wasp extracts prepared in Example 1 were measured. For measurement of the total polyphenol content, 50 μl of Folin-ciocalteu and 100 μl of Na₂CO₃ saturated solution were added to 400 μl of each extract sample and allowed to stand at room temperature for 1 hour, and then the absorbance at 725 nm was measured. As a standard reagent, tannic acid was used. For measurement of the total flavonoid content, each extract sample was extracted by stirring with methanol for 18 hours and filtered, and 4 ml of 90% diethylene glycol was added to each of the filtrate samples and 40 μl of 1N NaOH was added thereto, and each of the mixtures was allowed to react at 37° C. for 1 hour, and then the absorbance at 420 nm was measured. As a standard reagent, rutin was used. Reducing sugar was quantified using a DNS method, and total sugar was quantified using a phenol-sulfuric acid method.

TABLE 1
Analysis of components of wasp extracts

| | Content (mg/g) | | | |
|---|---|---|---|---|
| | Total | Total | | Reducing |
| Division | polyphenol | flavonoid | Total sugar | sugar |
| V. mandarinia | 33.5 ± 0.9 | 3.5 ± 0.1 | 83.0 ± 6.3 | 54.8 ± 0.0 |
| Smith | | | | |
| Vespa velutina | 40.1 ± 0.1 | 8.0 ± 2.5 | 115.2 ± 4.8 | 60.6 ± 11.5 |
| nigrithorax | | | | |
| V. simillima | 29.8 ± 3.8 | 4.2 ± 0.4 | 121.2 ± 3.3 | 53.6 ± 1.6 |
| simillima Smith | | | | |

As a result, as shown in Table 1 above, the extracts of the three wasp species showed a total polyphenol content of 29.8 to 40.1 mg/g, and the Vespa velutina nigrithorax extract showed the highest total polyphenol content. The total flavonoid content was 3.5 to 8.0 mg/g and was the highest in the Vespa velutina nigrithorax extract. However, the total sugar content was the highest in the V. simillima simillima Smith extract (121.1 mg/g), which was 1.46 times higher than that in the V. mandarinia Smith extract, and the reducing sugar content was 53.6 to 60.6 mg/g and did not significantly differ between the extracts.

### Example 3: Evaluation of Blood Clotting Inhibitory Activities of Wasp Extracts

The blood clotting inhibitory activity (thrombus formation inhibitory activity) of each of the wasp extracts prepared in Example 1 was evaluated by measuring the thrombin time, the prothrombin time and the aPTT time in the same manner as a previously reported method (Sohn et al., 2004. Kor. J. Pharmacogn 35. 52-61; Kwon et al., 2004. J. Life Science, 14. 509-513; Ryu et al., 2010. J. Life Science, 20. 922-928). As plasma, control plasma (MD Pacific Technology Co., Ltd., Huayuan Industrial Area, China) was used. The thrombin time, the prothrombin time and the aPTT time were measured by the following methods.

Thrombin Time

50 μl of 0.5 U thrombin (Sigma Co., USA), 50 μl of 20 mM CaCl₂) and 10 μl of each sample extract at various concentrations were mixed in a tube of Amelung coagulometer KC-1A (Japan) at 37° C. and allowed to react for 2 minutes, and then 100 μl of plasma was added thereto and the time taken for the plasma to clot was measured. As a control, aspirin (Sigma Co., USA) was used, and a solvent control, DMSO was used instead of the sample. DMSO showed a clotting time of 30.5 seconds. The thrombin inhibitory effect was expressed as the average of three or more repeated experiments, and the thrombin inhibitory activity was expressed as the value obtained by dividing the clotting time after sample addition by the clotting time of the solvent control.

Prothrombin Time

70 μl of standard plasma (MD Pacific Co., China) and 10 μl of each sample extract at various concentrations were added to a tube of Amelung coagulometer KC-1A (Japan) and warmed at 37° C. for 3 minutes, and then 130 μl of PT reagent was added thereto. The time taken for the plasma to clot was expressed as the average of three repeated experiments. As a control, aspirin (Sigma Co., USA) was used, and as a solvent control, DMSO was used instead of the sample. DMSO showed a clotting time of 16.7 seconds. The prothrombin inhibitory activity was expressed as the value obtained by dividing the clotting time after sample addition by the clotting time of the solvent control.

Activated Partial Thromboplastin Time (aPTT)

100 μl of plasma and 10 μl of each sample extract at various concentrations were added to a tube of Amelung coagulometer KC-1A (Japan) and warmed at 37° C. for 3 minutes, and then 50 μl of aPTT reagent (Sigma, ALEXIN™) was added thereto, followed by incubation at 37° C. for 3 minutes. Thereafter, 50 μl of CaCl₂) (35 mM) was added thereto, and then the time taken for the plasma to clot was measured. As a solvent control, DMSO was used instead of the sample and showed a clotting time of 58.1 seconds. The aPTT was expressed as the average of three repeated experiments, and the blood clotting inhibitory activity was expressed as the value obtained by dividing the aPTT after sample addition by the aPTT of the solvent control.

TABLE 2
Evaluation of blood clotting inhibitory activity
of Vespa velutina nigrithorax extract

| | | Antithrombotic activity (blood clotting | | |
|---|---|---|---|---|
| | | time after sample addition/blood | | |
| | | clotting time after solvent addition | | |
| | | | | Blood clotting |
| | | | | factor |
| | | | | inhibition |
| | Concentration | Thrombin | Prothrombin | time |
| Division | (mg/ml) | time | time | (aPTT time) |
| DMSO | — | 1.00 ± 0.00 | 1.00 ± 0.00 | 1.00 ± 0.00 |
| Aspirin | 1.5 | 1.54 ± 0.11 | 1.44 ± 0.04 | 1.55 ± 0.07 |
| V. mandarinia | 5.0 | 1.09 ± 0.04 | 1.07 ± 0.05 | 1/27 ± 0.08 |
| Smith | | | | |
| Vespa velutina | 5.0 | 1.27 ± 0.06 | 1.22 ± 0.06 | 1.35 ± 0.10 |
| nigrithorax | | | | |
| V simillima | 5.0 | 1.09 ± 0.05 | 1.12 ± 0.06 | 1.19 ± 0.01 |
| simillima | | | | |
| Smith | | | | |

As a result, as shown in Table 2, it was confirmed that the wasp extracts had anticoagulant activity, and in particular, had an excellent effect of prolonging the aPTT time by inhibiting blood clotting factors. Among the extracts of the three wasp species, the Vespa velutina nigrithorax extract exhibited an excellent anticoagulant activity. As a result of measuring the thrombin time, the prothrombin time and the aPT time after adding the ethanol extract at a concentration of 5 mg/ml, the ethanol extract exhibited the effect of prolonging the thrombin time, the prothrombin time and the aPT time by 1.27-fold, 1.22-fold and 1.35-fold, respectively, compared to DMSO. Aspirin (trade name: Protect), which is a commercial antithrombotic agent used as a control, prolonged the thrombin time, the prothrombin time and the aPT time by 1.54-fold, 1.44-fold and 1.55-fold, respectively, at a concentration of 1.5 mg/ml, compared to DMSO, and thus it was considered that the Vespa velutina nigrithorax extract would exhibit an excellent antithrombotic effect.

### Example 4: Preparation and Component Analysis of Sequential Organic Solvent Fractions of Ethanol Extract of Vespa velutina nigrithorax Extract

Through Example 3, it was confirmed that the Vespa velutina nigrithorax extract had excellent anticoagulant activity. Thus, the Vespa velutina nigrithorax extract was fractionated sequentially with organic solvents (hexene, ethyl acetate and butanol), and water residue was finally recovered. Table 3 below shows the results of analyzing organic solvent fractionation efficiency and the components of the fractions.

TABLE 3
Preparation and Component Analysis of Sequential Organic
Solvent Fractions of Ethanol Extract of Vespa velutina
nigrithorax Extract

| | | | Content (mg/g) | | | |
|---|---|---|---|---|---|---|
| | | Fractionation | Total | Total | Total | Reducing |
| Division | | yield (%) | polyphenol | flavonoid | sugar | sugar |
| Vespa | Hexene fraction | 0.9 | 22.9 ± 0.7 | 7.5 ± 0.1 | 35.7 ± 0.6 | 5.6 ± 0.1 |
| velutina | Ethyl acetate | 12.5 | 26.0 ± 1.6 | 12.0 ± 0.1 | 136.3 ± 11.2 | 6.9 ± 0.6 |
| nigrithorax | fraction | | | | | |
| extract | Butanol fraction | 53.2 | 54.1 ± 2.7 | 4.5 ± 0.6 | 116.4 ± 0.1 | 13.8 ± 2.4 |
| | Water residue | 33.4 | 40.8 ± 1.6 | 4.0 ± 0.3 | 105.1 ± 2.1 | 20.4 ± 0.7 |

As shown in Table 3 above, it could be seen that 53.24% of the Vespa velutina nigrithorax extract was fractionated into the butanol fraction and 33.4% of the extract was recovered as water residue, suggesting that most of the extract was a water-soluble component. In addition, the ethyl acetate fraction of the extract accounted for 12.5%. As a result of measuring the total polyphenol content of each fraction, the total polyphenol content was the highest in the butanol fraction (54.1 mg/g), was 40.8 mg/g in the water residue, and was relatively low in the ethyl acetate fraction (26.0 mg/g). However, as a result of analyzing the total flavonoid content, the total flavonoid content was the highest in the ethyl acetate fraction (12.0 mg/g). The total sugar content was the highest in the ethyl acetate fraction (136.3 mg/g), and the reducing sugar content was the highest in the water residue (20.4 mg/g). Therefore, it was confirmed that the ethyl acetate fraction contained large amounts of compounds having polyphenol/flavonoid structures to which saccharides are bound.

### Example 5: Evaluation of Blood Clotting Inhibitory Activities of Fractions of Vespa velutina Nigrithorax Extract

The blood clotting inhibitory activities of the fractions of the Vespa velutina nigrithorax extract, prepared in Example 4, were measured according to the same method as the antithrombotic activity evaluation described in Example 3. As a result, the ethyl acetate fraction exhibited blood clotting inhibition by strong inhibition of thrombin, prothrombin and blood clotting factors, and prolonged the thrombin time, the prothrombin time and the aPT time by 15-fold, 4.1-fold and 3.29-fold, respectively, at a concentration of 5 mg/ml, compared to DMSO, suggesting that it exhibited stronger anticoagulant activity than aspirin (1.5 mg/ml). In particular, the ethyl acetate fraction prolonged all the thrombin time, the prothrombin time and the aPT time by 15-fold or more at a concentration of 6 mg/ml compared to DMSO, suggesting that it exhibited strong anticoagulant activity in a concentration-dependent manner. These results indicate that the ethyl acetate fraction has significant antithrombotic activity even when compared to aspirin (trade name: Protect) which is a currently commercially available antithrombotic agent, and the ethyl acetate fraction of the Vespa velutina nigrithorax extract may be developed as a practical antithrombotic agent in the future.

TABLE 4
Evaluation of Blood Clotting Inhibitory Activities of Fractions of
Vespa velutina nigrithorax Extract

| | | Antithrombotic activity (blood | | | |
|---|---|---|---|---|---|
| | | clotting time after sample addition/blood | | | |
| | | clotting time after solvent addition) | | | |
| | | | | | Blood clotting |
| | | | | | factor |
| | | Concentration | Thrombin | Prothrombin | inhibition time |
| Division | | (mg/ml) | time | time | (aPT time) |
| DMSO | | — | 1.00 ± 0.04 | 1.00 ± 0.02 | 1.00 ± 0.02 |
| Aspirin | | 1.5 | 1.43 ± 0.04 | 1.73 ± 0.01 | 1.42 ± 0.03 |
| Vespa velutina | Hexene fraction | 5 | 1.10 ± 0.05 | 1.09 ± 0.06 | 1.11 ± 0.05 |
| nigrithorax | Ethyl acetate | 7 | >15 | >15 | >15 |
| extract | fraction | 6 | >15 | >15 | >15 |
| | | 5 | >15 | 4.10 ± 0.19 | 3.29 ± 1.15 |
| | Butanol fraction | 7 | 2.61 ± 0.22 | 1.65 ± 0.05 | 2.45 ± 0.13 |
| | | 6 | 1.83 ± 0.06 | 1.53 ± 0.18 | 1.85 ± 0.15 |
| | | 5 | 1.65 ± 0.10 | 1.15 ± 0.04 | 1.28 ± 0.28 |
| | Water residue | 5 | 1.34 ± 0.08 | 3.22 ± 0.22 | 0.97 ± 0.03 |

### Example 6: Human Platelet Aggregation Inhibitory Activities of Fractions of Vespa velutina Nigrithorax Extract

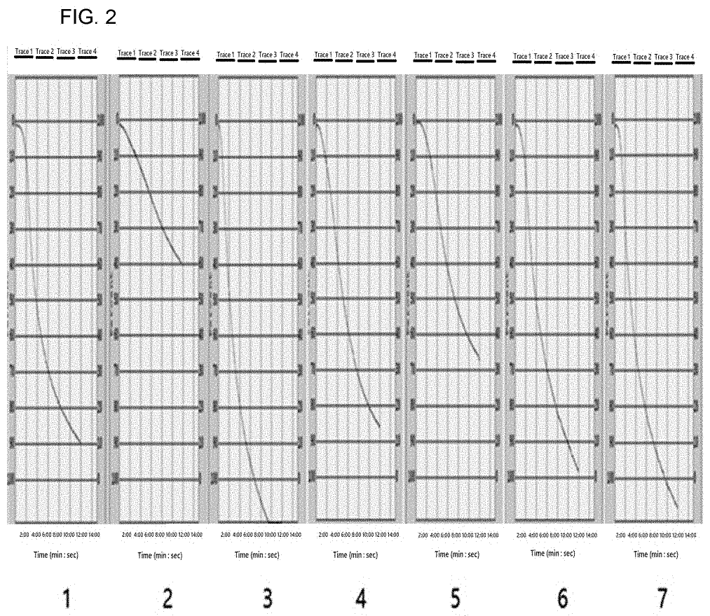

The human platelet aggregation inhibitory activities of the fractions of the Vespa velutina nigrithorax extract, prepared in Example 4, were measured, and the results of the measurement are shown in Table 5 below and FIG. 2. Platelets are disk-shaped small cells that circulate in blood vessels together with various blood cells. Instead of having a nucleus, platelets have cytoplasmic granules containing high concentrations of various substances related to protection from vascular damage and platelet aggregation. Platelets are important cells that secrete aggregation factors upon the occurrence of damage to the vascular inner wall, and bind to collagen exposed due to endothelial cell damage to form a primary hemostatic plug, thereby initiating thrombus formation. Thus, platelet aggregation inhibition is a very important activity to prevent thrombus formation. The platelet aggregation inhibitory activity of each fraction was evaluated according to the following method.

Platelet Aggregation Inhibitory Activity

As platelets, human concentrated plates were used, which were washed once with washing buffer (138 mM NaCl, 2.7 mM KCl, 12 mM NaHCO₃, 0.36 mM NaH₂PO₄, 5.5 mM glucose, 1 mM EDTA, pH 6.5). Thereafter, the platelets were resuspended in suspending buffer (138 mM NaCl, 2.7 mM KCl, 12 mM NaHCO₃, 0.36 mM NaH₂PO₄, 5.5 mM glucose, 0.49 mM MgCl₂, 0.25% gelatin, pH 7.4), and then centrifuged at 3,000 rpm for 10 minutes and resuspended again in suspending buffer. At this time, the number of the platelets was adjusted to 4×10⁹/ml. Thereafter, 2.5 μl of collagen was added to 1 ml of the suspension and allowed to react for 5 minutes, and platelet aggregation at 37° C. was measured using a whole-blood aggregometer (Chrono-log, USA).

TABLE 5
Human Platelet Aggregation Inhibitory Activities
of Fractions of Vespa velutina nigrithorax Extract

| | | Platelet aggregation curve | | | | |
|---|---|---|---|---|---|---|
| | | | | | Area | Platelet |
| | Concentration | Amplitude | | Lag time | under | aggregation |
| Sample/control | (mg/ml) | (ohm) | Slope | (seconds) | curve | rate (%) |
| Solvent control | — | 22 | 6 | 62 | 168.5 | 100.0 |
| (DMSO) | | | | | | |
| Aspirin | 0.25 | 10 | 1 | 56 | 59.1 | 35.1 |
| Hexene fraction | 0.25 | 21 | 3 | 38 | 147.4 | 87.5 |
| Ethyl acetate | 0.25 | 17 | 3 | 72 | 107.5 | 63.8 |
| fraction | | | | | | |
| Butanol fraction | 0.25 | 24 | 5 | 42 | 175.8 | 104.3 |
| Water residue | 0.25 | 27 | 6 | 34 | 207.7 | 123.3 |

As a result, it was confirmed that the hexene and ethyl acetate fractions exhibited platelet aggregation inhibitory activity at a concentration of 0.25 mg/ml, whereas the butanol fraction and the water residues had no effect on platelet aggregation or rather promoted platelet aggregation. In particular, the ethyl acetate fraction showed a platelet aggregation rate of 63.8% of the solvent control at a concentration of 0.25 mg/ml, and thus exhibited a platelet aggregation inhibitory activity corresponding to ½ of that of aspirin. Therefore, it was confirmed that the ethyl acetate fraction of the Vespa velutina nigrithorax extract exhibited platelet aggregation inhibitory activity while exhibiting excellent anticoagulant activity, suggesting that it can be used as a practical antithrombotic agent.

### Example 7: Evaluation of Erythrocyte Hemolytic Activity of Wasp Extract

In order to evaluate the acute toxicities of the Vespa velutina nigrithorax extract and the fractions thereof, the human erythrocyte hemolytic activities thereof were evaluated. The results of the evaluation are shown in Table 6 below. At this time, the hemolytic activity was evaluated according to a previous report (Son Ho-Yong et al., 2014, Korean J. Microbiol. Biotechnol. 42: 285-292). Briefly, 100 μl of human erythrocytes were washed three times with PBS and added to a 96-well microplate, and 100 μl of a sample solution at various concentrations was added thereto and then allowed to react at 37° C. for 30 minutes. Thereafter, the reaction solution was centrifuged at 1,500 rpm for 10 minutes, and 100 μl of the supernatant was transferred to a fresh microtiter plate, and then the degree of hemoglobin leakage following hemolysis was measured at 414 nm. DMSO (2%) was used as a solvent control for the sample, and Triton X-100 (1 mg/ml) was used as an experimental control for hemolysis of erythrocytes. Hemolytic activity was calculated using the following equation.

(%)Hemolysis=[(Abs. S−Abs. C)/(Abs. T−Abs. C)]×100

Abs. S: absorbance of sample-added group

Abs. C: absorbance of DMSO-added group

Abs. T: absorbance of Triton X-100-added group

TABLE 6
Human erythrocyte hemolytic activities of Vespa velutina
nigrithorax extract and fractions thereof

| | | | Human | |
|---|---|---|---|---|
| | | | erythrocyte | |
| | | Concentration | hemolytic | |
| Sample/control | | (mg/ml) | activity | |
| Distilled water | | — | 0.0 | ± 0.3 |
| Solvent (DMSO) | | — | 1.5 | ± 1.0 |
| Triton X-100 | | 1.0 | 100.0 | ± 0.1 |
| Amphotericin B | | 0.1 | 95.8 | ± 0.3 |
| | | 0.05 | 72.4 | ± 5.2 |
| | | 0.025 | 59.5 | ± 2 |
| | | 0.0125 | 48.2 | ± 5.9 |
| | | 0.0063 | 21.6 | ± 3.7 |
| | | 0.0032 | 6.5 | ± 0.9 |
| | | 0 | 0.0 | ± 1.7 |
| Vespa velutina | Ethanol extract | 1.0 | 0.7 | ± 0.7 |
| nigrithorax | Hexene fraction | 1.0 | 2.0 | ± 0.5 |
| | Ethyl acetate fraction | 1.0 | −2.5 | ± 0.2 |
| | Butanol fraction | 1.0 | 0.1 | ± 0.6 |
| | Water residue | 1.0 | 1.1 | ± 0.1 |

As shown in Table 6 above, it was confirmed that DMSO and water used as controls had no hemolytic activity, and Triton X-100 hemolyzed 100% of erythrocytes at a concentration of 1 mg/ml. Amphotericin B, which is used as an anticancer agent, hemolyzed 59% or more of erythrocytes at a concentration of 0.025 mg/ml. Meanwhile, the Vespa velutina nigrithorax extract and the sequential organic solvent fractions thereof showed no erythrocyte hemolytic activity even at a concentration of 1.0 mg/ml. Thus, it is expected that the ethyl acetate fraction of the Vespa velutina nigrithorax extract will show no separate acute toxicity.

### Example 8: Evaluation of Plasma, Acid and Heat Stabilities of Ethyl Acetate Fraction of Vespa velutina nigrithorax Extract

The heat stability and acid stability for antithrombotic activity of the ethyl acetate fraction obtained in Example 4 were evaluated. The fraction maintained excellent activity without significant loss of its anticoagulant activity and platelet aggregation inhibitory activity even when it was heat-treated at 100° C. for 1 hour or treated at pH 2 (0.01 M HCl) for 1 hour. Therefore, it is expected that the ethyl acetate fraction of the Vespa velutina nigrithorax extract will maintain its heat resistance and acid resistance even in various food processing processes.

As demonstrated through the Examples of the present disclosure, the Vespa velutina nigrithorax extract as an active ingredient for a pharmaceutical composition and a health functional food for preventing, ameliorating or treating thrombosis according to the present disclosure exhibits antithrombotic activity by inhibiting platelet aggregation while exhibiting strong anticoagulant activity by inhibiting thrombosis-related enzymes and blood clotting factors. In addition, the antithrombotic active component of the Vespa velutina nigrithorax extract has no human erythrocyte hemolytic activity, has excellent heat stability, and the thrombosis-related enzyme and blood clotting factor inhibitory effects thereof are not lost even under an acidic condition of pH 2 and in plasma. Thus, it is expected that the antithrombotic active ingredient of the Vespa velutina nigrithorax extract can be used to prevent and treat thrombotic diseases such as ischemic stroke and hemorrhagic stroke by improving blood circulation. In addition, the active ingredient has an excellent effect in that it may be processed into various forms such as extracts, powders, pills or tablets, which can be taken at all times. Thus, the active ingredient is very useful in the pharmaceutical and food industries.

What is claimed is:

1. A pharmaceutical composition for preventing or treating thrombosis containing an extract of Vespa velutina nigrithorax as an active ingredient.

2. The pharmaceutical composition of claim 1, wherein the extract is an ethanol extract.

3. The pharmaceutical composition of claim 1, wherein the extract is an ethyl acetate fraction obtained after ethanol extraction.

4. A health functional food for preventing or ameliorating thrombosis containing an extract of Vespa velutina nigrithorax as an active ingredient.

5. The health functional food of claim 4, wherein the extract is an ethanol extract.

6. The health functional food of claim 4, wherein the extract is an ethyl acetate fraction obtained after ethanol extraction.
